(19) United States
(12) Patent Application Publication
(10) Pub. No.: US 2019/0000346 A1
(43) Pub. Date: Jan. 3, 2019

(54) SKIN CONDUCTANCE-BASED REGULATION OF AN ELECTRONIC DEVICE

(71) Applicant: THOMSON Licensing, Issy-les-Moulineaux (FR)

(72) Inventors: Brian Charles Eriksson, San Jose, CA (US); Ajith Pudhiyaveetil, Sunnyvale, CA (US); Yifan Sun, Mountain View, CA (US); Weicong Ding, Sunnyvale, CA (US); Can Karakus, Los Altos, CA (US)

(21) Appl. No.: 16/022,582

(22) Filed: Jun. 28, 2018

Related U.S. Application Data
(60) Provisional application No. 62/526,572, filed on Jun. 29, 2017.

(51) Int. Cl. A61B 5/053; A61B 5/01; A61B 5/00

(52) U.S. Cl. CPC A61B 5/0533; A61B 5/01; A61B 5/7225; A61B 5/6804; A61B 5/7203; A61B 5/6824; A61B 5/0004

(57) ABSTRACT

A system and method for skin conductance-based regulation of an electronic device is described. The system includes a processor configured to receive, data representative of skin conductance of a user; determine, a conductance for the user as a function of a temperature and the skin conductance; and adjust, the temperature based on the determined conductance.

### REFERENCE TO RELATED APPLICATION

This application claims priority from U.S. Provisional Application No. 62/526,572, entitled “SKIN CONDUCTANCE-BASED REGULATION OF AN ELECTRONIC DEVICE”, filed on Jun. 29, 2017, the contents of which are hereby incorporated by reference in its entirety.

### FIELD

The proposed apparatus is directed to Galvanic Skin Response (GSR) data and controlling an electronic device based on GSR data.

### BACKGROUND

This section is intended to introduce the reader to various aspects of art, which may be related to embodiments that are described below. This discussion is believed to be helpful in providing the reader with background information to facilitate a better understanding of the various aspects of the present disclosure. Accordingly, it should be understood, that these statements are to be read in this light.

Galvanic Skin Response (GSR) signals constitute a measure of skin conductance. Measurement of GSR signals occur by placing two electrodes on the skin of a user, then applying a very small voltage across the electrodes and measuring the current passing through the skin. More current means higher conductance, thus establishing the GSR signal.

The traditional theory of GSR holds that skin conductance varies with the state of sweat glands in the skin. Sweating is controlled by the sympathetic nervous system, and skin conductance is an indication of psychological or physiological arousal. If the sympathetic branch of the automatic nervous system is highly aroused, then sweat gland activity also increases. The sweat contains electrolytes which increases the conductance of the skin. Conversely, the thermoregulation of the human body through sweat glands ensures that skin temperature does not change greatly with ambient temperature.

Comfortable regulation of home temperature is a difficult problem. Some approaches that have been used include machine learning of patterns (Nest) or remote temperature monitoring (Honeywell). However, neither approach takes into consideration the user's perceived temperature of the environment.

Accordingly, there is a need for controlling home temperature based on a person's perceived temperature of the environment.

### SUMMARY

The proposed method and system concern Galvanic Skin Response (GSR) data and controlling an electronic device based on GSR data.

According to a first aspect of the disclosure a method is disclosed for receiving data representative of skin conductance of a user; determining a conductance for the user as a function of a temperature and the data representative of the skin conductance; and then adjusting the temperature based on the determined conductance.

In another embodiment, the data representative of skin conductance is received from a remote device.

In another embodiment, the remote device is one of a wrist device, a device attached to clothing, a device in furniture and a device in a remote control.

In another embodiment, the conductance for the user is determined by averaging the received data representative of the user's skin conductance for a period of time, as a function of temperature.

In another embodiment, the step of determining the conductance for the user further comprises: filtering the received data representative of skin conductance of the user by subtracting consecutive incoming data samples from each other; optimizing a user portion and a baseline portion of the filtered received data to recover non-zero user portions; and associating the non-zero user portions with the temperature.

In another embodiment, the filtering step includes one of removing noise from the received data and multiplying received data samples by a difference matrix.

In another embodiment, the step of adjusting the temperature based on the determined conductance further comprises: comparing the determined conductance to one of a default value or a historical user value indicative of a user discomfort level with the temperature; and instructing a temperature controller to modify the temperature based on the user discomfort level.

According to a second aspect of the disclosure, there is provided a system comprising a processor configured to, receive, data representative of skin conductance of a user; determine, a conductance for the user as a function of a temperature and the data representative of the skin conductance; and adjust, the temperature based on the determined conductance.

In another embodiment, the data representative of skin conductance is received from a remote device.

In another embodiment, the remote device is one of a wrist device, a device attached to clothing, a device in furniture and a device in a remote control.

In another embodiment, the conductance for the user is determined by averaging the received data representative of the user's skin conductance for a period of time as a function of temperature.

In another embodiment, the processor is further configured to: filter, the received data representative of skin conductance of the user by subtracting consecutive incoming data samples from each other; optimize, a user portion and a baseline portion of the filtered received data to recover non-zero user portions; and associate, the non-zero user portions with the ambient temperature.

In another embodiment, the received data is filtered by removing noise from the received data or by multiplying received data samples by a difference matrix.

In another embodiment, the processor is further configured to: compare, the determined conductance to one of a default value or a historical user value indicative of a user discomfort level with the temperature; and instruct a temperature controller to modify the temperature based on the user discomfort level.

Some processes implemented by elements of the disclosure may be computer implemented. Accordingly, such elements may take the form of an entirely hardware embodiment, an entirely software embodiment (including firmware, resident software, microcode, etc.) or an embodiment combining software and hardware aspects that may all generally be referred to herein as “circuit”, “module” or “system”. Furthermore, such elements may take the form of a computer program product embodied in any tangible medium of expression having computer usable program code embodied in the medium.

Since elements of the present disclosure can be implemented in software, the present disclosure can be embodied as computer readable code for provision to a programmable apparatus on any suitable carrier medium. A tangible carrier medium may comprise a storage medium such as a floppy disk, a CD-ROM, a hard disk drive, a magnetic tape device or a solid-state memory device and the like. A transient carrier medium may include a signal such as an electrical signal, an optical signal, an acoustic signal, a magnetic signal or an electromagnetic signal, e.g. a microwave or RF signal.

### BRIEF DESCRIPTION OF THE DRAWINGS

Embodiments of the disclosure will now be described by way of example only, and with reference to the following drawings in which:

FIG. 1 illustrates a flowchart of an exemplary method in accordance with one embodiment of the disclosure;

FIG. 2 depicts a device to generate Galvanic Skin Response (GSR) conductance data;

FIG. 3 depicts an exemplary graph of GSR conductance data from the device of FIG. 2 is plotted as a function of time;

FIG. 4 depicts a portion of the GSR conductance data from FIG. 3;

FIG. 5 depicts a block diagram of a system for processing the GSR conductance data representative of skin conductance of a user; and

FIG. 6 depicts a method for obtaining the processed GSR conductance data representative of skin conductance of a user.

### DETAILED DESCRIPTION

The proposed method and system is directed to Galvanic Skin Response (GSR) data and controlling an electronic device based on GSR data.

According to a first aspect of the disclosure a method is disclosed for receiving data representative of skin conductance of a user, determining a conductance for the user as a function of temperature and the data representative of the skin conductance and then adjusting the ambient temperature based on the determined conductance.

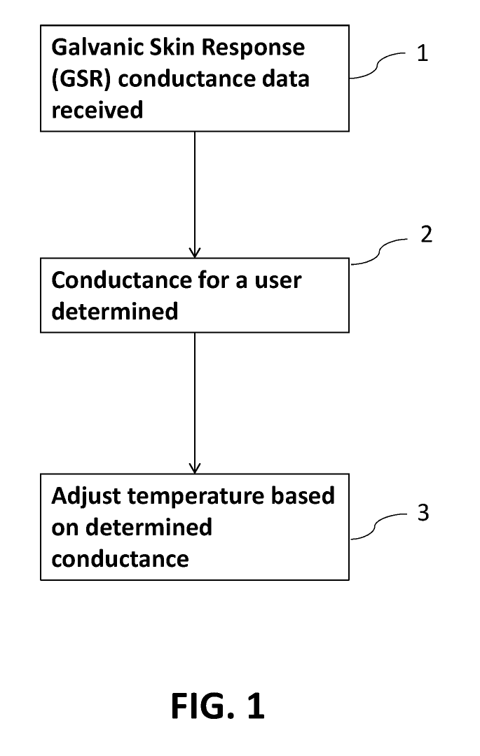

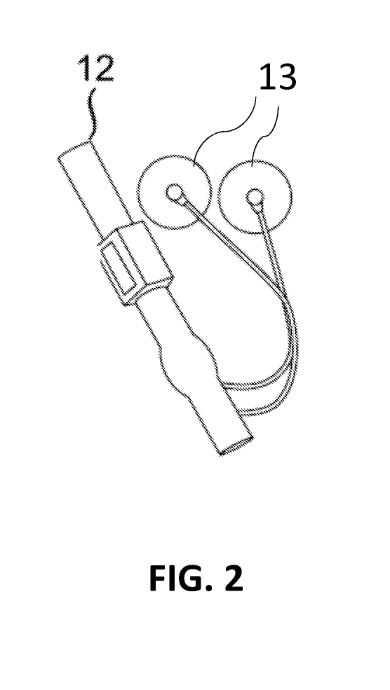

FIG. 1 illustrates a flowchart of an exemplary method in accordance with a first aspect of the disclosure. In step 1, data representative of skin conductance of a user is received from a remote device. Referring to FIG. 2, the remote device may be a wrist device 12. The wrist device 12 has two electrodes 13. Measurement of GSR conductance data occurs by placing the two electrodes 13 on the skin of a user, then applying a very small voltage across the electrodes and measuring the current passing through the skin.

The wrist device 12 transmits the GSR conductance data to a processor (not shown) configured to receive the GSR data. Alternatively, the remote device may be a device attached to clothing, a device in furniture and/or a device in a remote control.

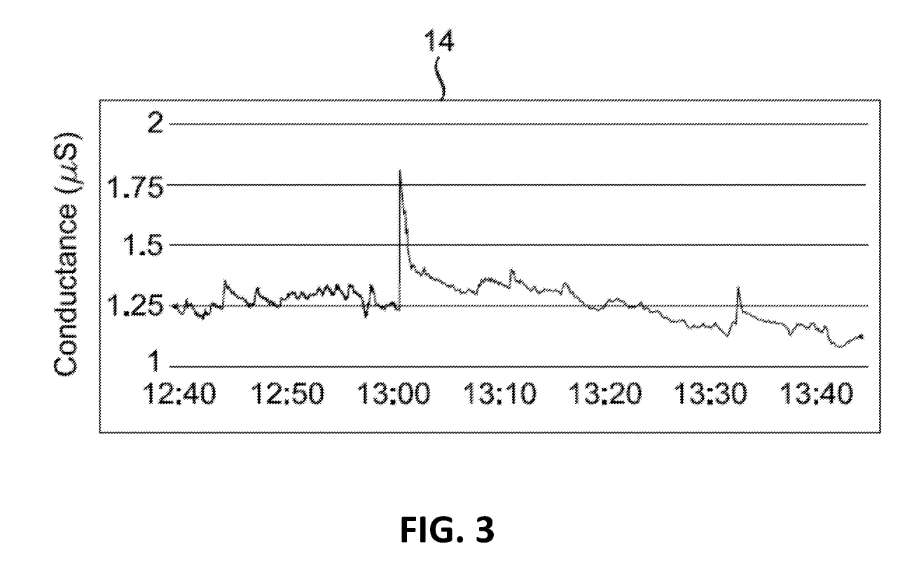

FIG. 3 shows an exemplary graph 14 of GSR conductance data plotted as a function of time received from the wrist device 12 shown if FIG. 2. The GSR conductance data depicted on the graph 14 will exhibit a correlation with the temperature within which the user is located.

Referring to step 2 of FIG. 1, the conductance for the user is determined based on the temperature and the data representative of skin conductance of the user. The conductance for the user is determined by removing data not associated with the temperature. Example of such data include instances of user excitement at the time of measurement. In one embodiment, data associated with instances of user excitement can be removed or minimized by averaging the received data representative of skin conductance of the user, for a period of time, as a function of temperature.

Alternatively, data not associated with temperature may be removed by first filtering the received data representative of skin conductance of the user. The filtering may be performed by subtracting consecutive incoming data samples from each other. Thereafter, optimizing a user portion and a baseline portion of the filtered received data to recover non-zero user portions; and finally associating the non-zero user portions with the temperature.

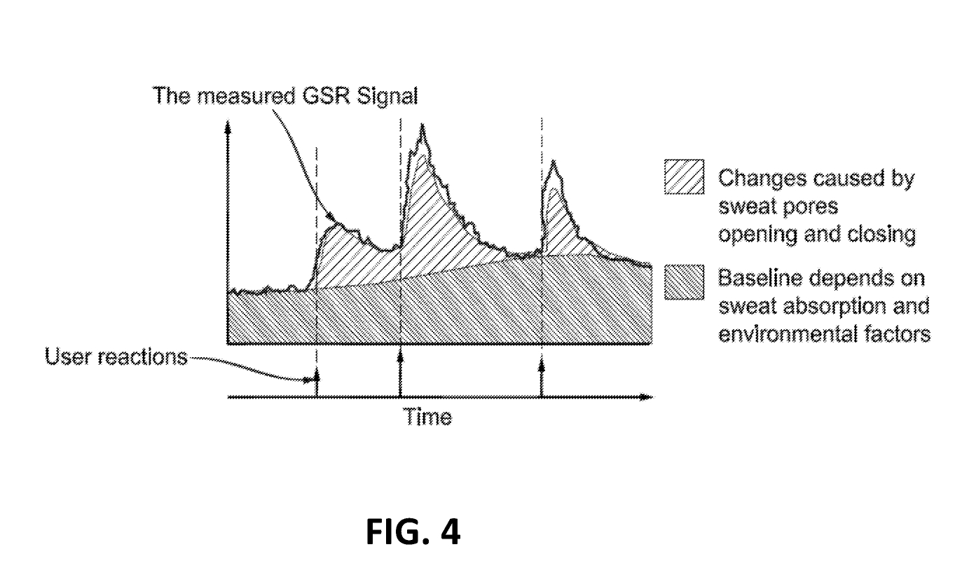

FIG. 4 depicts an enlargement of the GSR conductance data depicted in the chart 14 of FIG. 3, illustrating various components of the user's response. As depicted in FIG. 4, noise in the GSR signals obfuscates the user's response. In accordance with the present principles, the GSR signal undergoes processing to identify user reactions in the GSR signal (shown by vertical arrows in FIG. 4) in response to the environment experienced by the user.

To better understand the GSR signal processing technique to remove data not associated with the temperature, consider the following notation. Suppose the content viewed by the user has a duration of ‘T’ seconds and sampling of the user's GSR signal occurs every second. Let ‘x’ constitute a ‘T’ dimensional vector representing the user's reactions to the content every second, and ‘h’ a ‘t’ dimensional vector (with t<<T) capturing the typical sweat response of the user. For the sake of simplicity, a Linear Time Invariant (LTI) system will model the user, with the impulse response ‘h’ representing the typical way the user sweats when the user finds something exciting in the content.

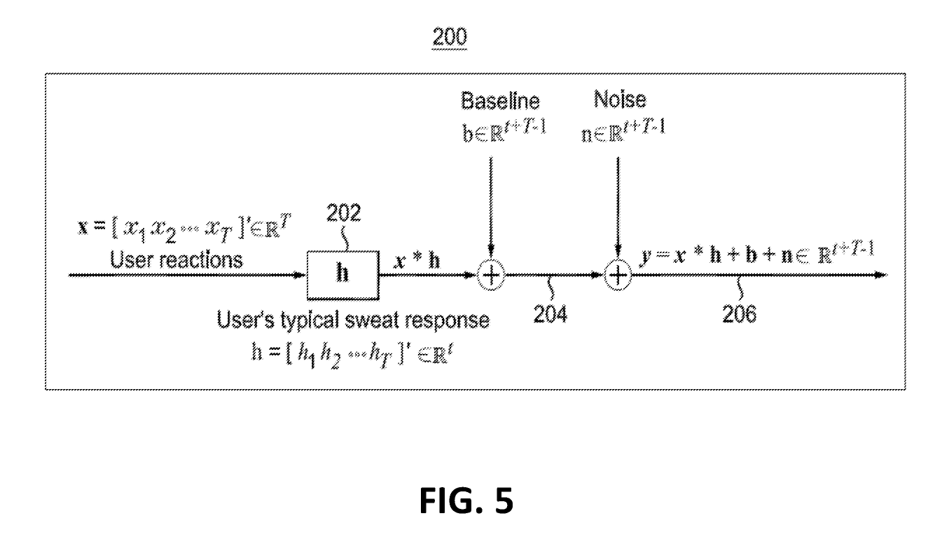

FIG. 5 depicts a block schematic diagram of a system 200 for obtaining GSR signal responses in accordance with an illustrative embodiment of the disclosure. The system 200 typically comprises a processor or computer that typically includes a central processing unit (CPU) (not shown) along with various peripheral devices (keyboard, mouse, display, network adapters) (not shown) along with a power supply (not shown). As described in greater detail hereinafter, the system 200 of FIG. 5 advantageously accounts for the GSR baseline signal ‘b’ and noise as ‘n’ in determining the final GSR signal response.

In FIG. 5, the system 200 includes a block 202 that performs the convolution of ‘x’ and ‘h’, represented by the term x*h′. The convolution operation performed by the block 202 can be represented as a matrix vector multiplication as follows:

$$( t + T - 1 ) \times T Tall Toeplitz Matrix$$

$$y = h * x = \underbrace{[ \begin{matrix}{h}_{1} & 0 & … & 0 \\ {h}_{2} & {h}_{1} & ⋮ & ⋮ \\ ⋮ & ⋮ & ⋱ & 0 \\ {h}_{t} & {h}_{t - 1} & ⋮ & {h}_{1} \\ 0 & {h}_{t} & & \\ ⋮ & ⋮ & ⋱ & \\ 0 & … & … & {h}_{t}\end{matrix} ]}_{{T}_{h}} [ \begin{matrix}{x}_{1} \\ ⋮ \\ {x}_{T}\end{matrix} ]$$

where the ‘Tₕ’ is a (t+T−t) by T tall Toeplitz matrix as shown above. With this the final observation ‘y’ can be written as

y=x*h+b+n=Tₕx+b+n

In accordance with an aspect of the present principles, the effect of the baseline signal is mitigated by filtering the observed signal such that the baseline component of the GSR signal does not obfuscate the user's response. Such filtering occurs by subtracting consecutive components from the observed GSR signal ‘y’ via block 204 in the system 200 in the following manner:

$$D = [ \begin{matrix}1 & - 1 & 0 & … & 0 \\ 0 & 1 & - 1 & … & 0 \\ ⋮ & ⋱ & ⋱ & … & 0 \\ 0 & … & … & 1 & - 1\end{matrix} ]$$

$$y = {T}_{h} x + b + n Dy = {DT}_{h} x + Db + Dn$$

The subtraction of consecutive samples of the observation can be achieved simply by multiplying the observation by the difference matrix ‘D’ shown above the arrow in above figure. The above-described matrix includes noise subtraction represented by the block 206.

After taking the difference of the consecutive samples and filtering out the noise, the user reactions ‘x’ part in the GSR signal and the transformed baseline ‘Db’ component of the GSR signal have same structure. Both ‘x’ and ‘Db’ are sparse. These observations enable solving the following optimization problem to obtain estimates of ‘x’:

$$x ∈ {T}^{} , {min}_{u ∈ {ℝ}^{T + t - 2}} {‖ x ‖}_{1} + {‖ u ‖}_{1}$$

$$subject to {‖ Dy - [ \begin{matrix}{DT}_{h} & I\end{matrix} ] [ \begin{matrix}x \\ u\end{matrix} ] ‖}_{2} \leq η$$

where x represents user reaction, u=Db represents the filtered baseline signal, Dy represents filtered observation, D represents a difference matrix and Tₕ represents Toeplitz matrix for user's typical sweat response and I represent the identity matrix.

The parameter ‘η’ represents the tuning parameter used to fine-tune the output. Standard open source numerical optimization software packages can be used to solve this problem easily.

The GSR signal processing technique described with respect to FIG. 5, makes use of a more realistic single model for GSR. In addition, such GSR signal processing technique yields an optimization problem much easier to solve than prior approaches. For example, prior approaches typically require many lines of code, while the technique described with respect to FIG. 5 can be implemented in 4 lines of code. In addition, the computation time required by this GSR signal processing is significantly faster than other approaches.

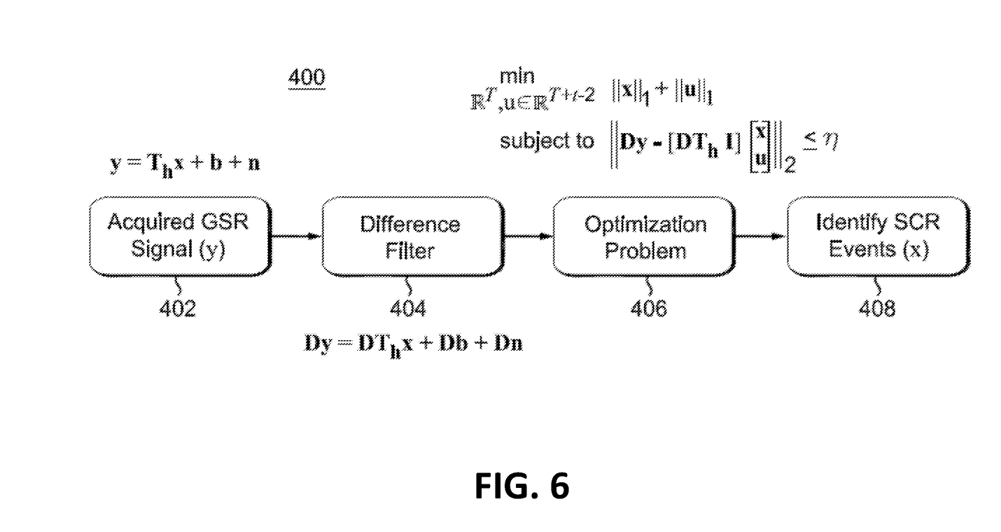

FIG. 6 depicts a flow chart of a method 400 in accordance with an embodiment of the disclosure for correlating GSR signals from a single user. During step 402 of FIG. 6, the system 200 of FIG. 5 acquires GSR signals for the user during step 402. Next, the user's GSR signals are filtered to subtract consecutive samples and remove noise during step 404 (representing, the operations performed by the blocks 202, 204, and 206 of FIG. 5). The user reaction portion and baseline portion of the GSR signal is optimized during step 406 to recover non-zero user responses at corresponding locations in the content. The optimization occurs by solving the following optimization problem described above.

$$x ∈ {T}^{} , {min}_{u ∈ {ℝ}^{T + t - 2}} {‖ x ‖}_{1} + {‖ u ‖}_{1}$$

$$subject to {‖ Dy - [ \begin{matrix}{DT}_{h} & I\end{matrix} ] [ \begin{matrix}x \\ u\end{matrix} ] ‖}_{2} \leq η$$

where x represents user reaction, u=Db represents the filtered baseline signal, Dy represents filtered observation, D represents a difference matrix and Tₕ represents Toeplitz matrix for the user's typical sweat response and I represent the identity matrix.

The locations in the content having the non-zero responses are then identified as times of interest during step 408 corresponding to the user reaction areas depicted by arrows in FIG. 4.

Referring to step 3 of FIG. 1, the determined conductance for the user based on the temperature is then used to adjust the temperature. The step of adjusting the temperature based on the determined conductance includes comparing the determined conductance to one of a default value or a historical user value indicative of a user discomfort level with the temperature and instructing a temperature controller to modify the temperature based on the user discomfort level. In one embodiment, the processor communicates with a thermostat to increase or decrease the temperature of the environment of the user indicative of the discomfort level thereof. Conversely, if the determined conductance for the user is not indicative of user discomfort, then the processor will not send instructions to the temperature controller for adjusting the temperature in the environment of the user.

It should be understood, that the elements shown in the figures may be implemented in various forms of hardware, software or combinations thereof. Preferably, these elements are implemented in a combination of hardware and software on one or more appropriately programmed general-purpose devices, which may include a processor, memory and input/output interfaces. Herein the phrase “coupled” is defined to mean directly connected to or indirectly connected with, through one or more intermediate components. Such intermediate components may include both hardware and software-based components.

The present description illustrates the principles of the present disclosure. It will thus be appreciated that those skilled in the art will be able to devise various arrangements that, although not explicitly described or shown herein, embody the principles of the disclosure and are included within its scope.

All examples and conditional language recited herein are intended for educational purposes to aid the reader in understanding the principles of the disclosure and the concepts contributed by the inventors to furthering the art, and, are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that the block diagram presented herein represents conceptual views of illustrative circuitry embodying the principles of the disclosure. Similarly, it will be appreciated that any flow charts, flow diagrams, state transition diagrams, pseudocode and the like represent various processes which may be substantially represented in computer readable media and so executed by a computer or processor, whether or not such computer or processor is explicitly shown.

The functions of the various elements shown in the figures may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, read only memory (ROM) for storing software, random access memory (RAM), and nonvolatile storage.

Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

In the claims, hereof, any element expressed as a means for performing a specified function is intended to encompass any way of performing that function including, for example, a) a combination of circuit elements that performs that function or b) software in any form, including, therefore, firmware, microcode or the like, combined with appropriate circuitry for executing that software to perform the function. The disclosure as defined by such claims resides in the fact that the functionalities provided by the various recited means are combined and brought together in the manner which the claims call for. It is thus regarded that any means that can provide those functionalities are equivalent to those shown herein.

1. A method, comprising:
receiving data representative of skin conductance of a user;
determining a conductance for the user as a function of a temperature and the data representative of the skin conductance; and
adjusting the temperature based on the determined conductance.

2. The method of claim 1, wherein the data representative of skin conductance is received from a remote device.

3. The method of claim 2, wherein the remote device is one of a wrist device, a device attached to clothing, a device in furniture and a device in a remote control.

4. The method of claim 1, wherein the conductance for the user is determined by averaging the received data representative of skin conductance of the user for a period of time as a function of temperature.

5. The method of claim 1, wherein the step of determining the conductance for the user further comprises:
filtering the received data representative of skin conductance of the user by subtracting consecutive incoming data samples from each other;
optimizing a user portion and a baseline portion of the filtered received data to recover non-zero user portions; and
associating the non-zero user portions with the temperature.

6. The method of claim 5, wherein the filtering step includes one of removing noise from the received data representative of skin conductance of the user and multiplying samples of received data representative of skin conductance of the user by a difference matrix.

7. The method of claim 1, wherein the step of adjusting the temperature based on the determined conductance further comprises:
comparing the determined conductance to one of a default value or a historical user value indicative of a user discomfort level with the temperature; and
instructing a temperature controller to modify the temperature based on the user discomfort level.

8. A system, comprising:
a processor configured to,
receive, data representative of skin conductance of a user;
determine, a conductance for the user as a function of a temperature and the skin conductance; and
adjust, the temperature based on the determined conductance.

9. The system of claim 8, wherein the data representative of skin conductance is received from a remote device.

10. The system of claim 9, wherein the remote device is one of a wrist device, a device in clothing, a device in furniture, a device in a remote control.

11. The system of claim 8, wherein the conductance for the user is determined by averaging the received data representative of skin conductance of the user for a first period of time as a function of temperature.

12. The system of claim 8, wherein the processor is further configured to:
filter, the received data representative of skin conductance of the user by subtracting consecutive incoming data samples from each other;
optimize, a user portion and a baseline portion of the filtered received data to recover non-zero user portions; and
associate, the non-zero user portions with the temperature.

13. The system of claim 12, wherein the received data representative of skin conductance of the user is filtered by removing noise therefrom or by multiplying samples thereof by a difference matrix.

14. The system of claim 8, wherein the processor is further configured to:
compare, the determined conductance to one of a default value or a historical user value indicative of a user discomfort level with the temperature; and
instruct a temperature controller to modify the temperature based on the user discomfort level.

15. The system of claim 8, further including a memory configured to store the data representative of skin conductance of the user.

16. A computer program product for a programmable apparatus, the computer program product comprising a sequence of instructions for implementing the method according to claim 1, when loaded into and executed by the programmable apparatus.

17. A non-transitory computer readable medium having one or more executable instructions stored thereon, which when executed by a processor cause the processor to perform the method according to claim 1.
